(19) United States
(12) Patent Application Publication
(10) Pub. No.: US 2004/0185470 A1
(43) Pub. Date: Sep. 23, 2004

(54) Nucleic acid detection

(76) Inventor: Robert C. Getts, Collegeville, PA (US)

Correspondence Address: Morris E. Cohen, Suite 217, 1122 Coney Island Avenue, Brooklyn, NY, 11230-2345, US

(21) Appl. No.: 10/730,823

(22) Filed: Dec. 8, 2003

Related U.S. Application Data
(63) Continuation of application No. 10/393,519, filed on Mar. 20, 2003, now abandoned.
Application No. 10/393,519 is a continuation of application No. PCT/US01/29589, filed on Sep. 20, 2001.
(63) Continuation-in-part of application No. 09/802,162, filed on Mar. 8, 2001.
(63) Continuation-in-part of application No. 09/908,950, filed on Jul. 19, 2001.
(60) Provisional application No. 60/234,060, filed on Sep. 20, 2000.
(60) Provisional application No. 60/187,681, filed on Mar. 8, 2000.
(60) Provisional application No. 60/219,397, filed on Jul. 19, 2000.

(51) Int. Cl. C12Q 1/68; C12P 19/34

(52) U.S. Cl. 435/6; 435/91.2

(57) ABSTRACT

Methods for detecting nucleic acids that do not require incorporation of labeled nucleotides into the hybridized strand of either the target or the probe nucleic acid. A nucleic acid is detected by adding a capture sequence onto the end of the single stranded probe or target and then hybridizing that capture sequence to a complementary sequence on a signal carrying molecule. Alternatively, a nucleic acid is detected by using an existing sequence on one of the two unlabeled single strands as the capture sequence, and then hybridizing that sequence to a complementary sequence of a signal carrying molecule. In preferred embodiments, the signal carrying molecule is a dendrimer.

### RELATED APPLICATIONS

The present application is a continuation of U.S. Nonprovisional Application Serial No. 10/393,519 filed Mar. 20, 2003, which is a continuation and claims the priority of PCT Application Ser. No. PCT/US01/29589 filed Sep. 20, 2001, which claims the priority of U.S. Provisional Application Serial No. 60/234,060 filed Sep. 20, 2000. The present application is also a continuation-in-part of U.S. Nonprovisional Application Serial No. 09/802,162 filed Mar. 8, 2001 which claims the priority of U.S. Provisional Application Serial No. 60/187,681 filed Mar. 8, 2000. The present application is also a continuation-in-part of U.S. Nonprovisional Application Serial No. 09/908,950 filed Jul. 19, 2001 which claims the priority of U.S. Provisional Application Serial No. 60/219,397 filed Jul. 19, 2000. The priority of all of those applications is claimed, all of which are fully incorporated herein by reference.

### FIELD OF THE INVENTION

The present invention is directed to methods for the detection of nucleic acids.

### BACKGROUND OF THE INVENTION

As is well known in the art, nucleic acid detection is traditionally performed by hybridizing two complementary strands of nucleic acid (DNA or RNA), one of which is the target molecule and one of which is the probe. Labeled nucleotides are incorporated into one of the two nucleic acid strands prior to hybridization. The detection of strands containing those labeled nucleotides allows detection and identification of the presence of particular nucleic acids in the sample.

Several different formats can be used in conjunction with the hybridization, i.e. liquid-phase hybridization, solid-phase hybridization, or in-situ hybridization on tissues or cell bodies. The kinetics associated with the different nucleic acid hybridization reactions have been well documented. See e.g., Britten et al., 1974, Methods Enzymol, 29, p. 363; Kohme et al., 1977 Biochemistry, 16 pp. 5329-5341.

Numerous labeling methods are also well known, but each has their disadvantages. For example, radioisotopes such as ³²P are frequently used to label nucleotides, with the radiolabeled nucleotide being incorporated into the nucleic acid chain. However, labeling with radioisotopes can generate large amounts of radioactive waste reagent, and can result in poor reproducibility and high background levels caused by nonspecific binding.

Biotin labeling is also common, but requires costly reagents and the need for extensive controls. Fluorescent labeling is another widely utilized technique, but interfering substances such as detergents, proteins, and lipids can affect the reproducibility of the signal when intercalating fluorescent dyes into nucleic acids.

Accordingly, it is desirable to introduce an improved method of nucleic acid detection to the art, as disclosed herein. As disclosed below, such a method is provided herein.

In certain embodiments, it would be further desirable to provide an improved method for nucleic acid detection that could be utilized in conjunction with microarrays and with dendritic molecules.

The microarray is a high-speed technology useful for DNA analysis. Microarrays include a plurality of distinct DNA or gene probes (i.e., polynucleotides) distributed spatially, and stably associated with a substantially planar substrate such as a plate of glass, silicon or nylon membrane. Such microarrays have been developed and are used in a range of applications such as analyzing a sample for the presence of gene variations or mutations (i.e. genotyping), or for patterns of gene expression, while performing the equivalent of thousands of individual “test-tube” experiments carried out in a short period of time.

Generally, microarrays operate on a similar principle: a substantially planar substrate such as a glass coverslide is coated with a grid of tiny spots of about 20 to 100 microns in diameter; each spot or feature contains millions of copies of a short sequence of DNA or nucleotides; and a computer keeps track of each sequence at a predetermined feature. To make an analysis, messenger RNA (mRNA) is extracted from a sample of cells. Using enzymes, millions of copies of the mRNA molecules are reproduced. Copies of complementary DNA (cDNA) are generated from the mRNA through reverse transcription. Currently, the cDNA copies are tagged with a marker or label such as a fluorescent marker and broken up into short fragments. The tagged fragments are washed over the microarray and left overnight, to allow the tagged fragments to hybridize with the DNA attached to the microarray.

After hybridization, the features on the microarray that have paired with the fluorescent cDNA emit a fluorescent signal that can be viewed with a microscope or detected by a computer. In this manner, one can learn which sequences on the microarray match the cDNA of the test sample. Although there are occasional mismatches, the employment of millions of probes in each spot or feature ensure fluorescence is detected only if the complementary cDNA is present. The more intense the fluorescent signal, (i.e. the brighter the spot) the more matching cDNA was present in the cell.

Dendritic nucleic acid molecules, or dendrimers, are complex, highly branched molecules, comprised of a plurality of interconnected natural or synthetic monomeric subunits of double-stranded DNA. Dendrimers are described in greater detail in U.S. Pat. Nos. 5,175,270 and 5,484,904, and in Nilsen et al., Dendritic Nucleic Acid Structures, J. Theor. Biol., 187, 273-284 (1997), all of which are fully incorporated herein by reference.

Dendrimers comprise two types of single-stranded hybridization “arms” on the surface which are used to attach two key functionalities. A single dendrimer molecule may have at least one hundred arms of each type on the surface. One type of arm is used for attachment of a specific targeting molecule to establish target specificity and the other is used for attachment of a label or marker (the signal). The molecules that determine the target and labeling specificities of the dendrimer are attached either as oligonucleotides or as oligonucleotide conjugates. Using simple DNA labeling, hybridization, and ligation reactions, a dendrimer molecule may be configured to act as a highly labeled, target specific probe.

The prepared mixture is formulated in the presence of a suitable buffer to yield a dendrimer hybridization mixture containing dendrimers with fluorescent labels attached to one type of “arm”, and with oligonucleotides attached to another type of “arm”, complementary to the capture sequences of the RT primer bound cDNA fragments. The dendrimer hybridization mixture containing the dendrimer molecules, is then added to the microarray and incubated overnight to generate a hybridization pattern. Subsequent to the dendrimer-to-cDNA hybridization, the microarray is washed to purge any excess unhybridized dendrimers. The microarray is scanned to detect the signal generated by the label to enable gene expression analysis of the hybridization pattern.

### SUMMARY OF THE INVENTION

It is an object of the present invention to provide improved methods of detecting nucleic acids.

It is a further object of the present invention to provide improved methods of detecting nucleic acids which can be used with conventional blot assays or other probe hybridization methods (e.g. in-situ hybridization).

It is a further object of the present invention to provide improved methods of detecting nucleic acids which can be used with microarrays.

Further objects of the invention will become apparent in conjunction with the disclosure provided herein.

To address the disadvantages of the prior art, methods are provided for detecting nudceic acids that do not require incorporation of labeled nucleotides into the hybridized strand of either the target or the probe nucleic acid. Accordingly, pursuant to the present inventions, a nucleic acid is detected by adding a capture sequence onto the end of the single stranded probe or target, both of which are unlabeled, and then hybridizing that capture sequence to a complementary sequence (“the probe binding sequence”) on a signal carrying molecule. Alternatively, a nucleic acid is detected by using an existing sequence on one of the two unlabeled single strands as the capture sequence, and then hybridizing that sequence to a complementary sequence of a signal carrying molecule. In the preferred embodiments of the invention, the signal carrying molecule is a dendrimer. Examples of preferred dendrimers are described in U.S. Pat. Nos. 5,175,270 and 5,484,904.

In one embodiment of the present invention, a method is provided which includes the steps of:

1) Providing RNA probes having an existing capture sequence attached thereto, or attaching a capture sequence to the probes;

2) Providing dendrimer molecules which (a) have a nucleic acid sequence complementary to the capture sequence (“the probe binding sequence”) and (b) have a signal molecule attached to the dendrimer;

3) Adding the RNA probes to the target nucleic acids, and allowing the probes to hybridize to any complementary strands of the target nucleic acids;

4) Adding dendrimer molecules to the probes, and allowing hybridization between the probes' capture sequences and the complementary, probe binding sequences on the dendrimers; and,

5) Detecting signal from dendrimers hybridized to the probes.

In a further embodiment of the present invention, a method is provided which includes the steps of:

1) Attaching target nucleic acid sequence to a support (e.g. a membrane);

2) Adding a capture sequence to RNA probes, or providing RNA probes having a preexisting capture sequence;

3) Providing dendrimer molecules which (a) have a nucleic acid sequence complementary to the capture sequence (“the probe binding sequence”) and (b) have a signal molecule attached to the dendrimer;

4) Adding the RNA probes to the target nucleic acids on the support, and allowing hybridization between the probes and any complementary strands of target nucleic acid;

5) Adding dendrimer molecules to the hybridized probes, and allowing hybridization between the probes' capture sequences and the complementary, probe binding sequences on the dendrimers; and,

6) Detecting signal from dendrimers hybridized to the probes.

In a further embodiment of the present invention, a method is provided which includes the steps of:

1) Adding an RT primer oligonucleotide to mRNA wherein the RT primer oligonucleotide includes a capture sequence therein (or wherein a capture sequence is subsequently added to the RT primer oligonucleotide);

2) Reverse transcribing the mRNA with unlabeled nucleotides (dATP, dTTP, dGTP and/or dCTP) to generate cDNA hybridized to the mRNA;

3) Degrading the mRNA strands leaving probes of single stranded cDNA that include the capture sequence;

4) Providing dendrimer molecules which (a) have a nucleic acid sequence complementary to the capture sequence (“the probe binding sequence”) and (b) have a signal molecule attached to the dendrimer;

5) Hybridizing the cDNA probes to a microarray containing target nucleic acid sequence;

6) Adding dendrimer molecules to the hybridized probes, and allowing hybridization between the capture sequence on the probes and the complementary, probe binding sequence on the dendrimers; and,

7) Detecting signal from dendrimers hybridized to the probes.

The above steps can be conducted in an order other than that disclosed above or as combined steps, while still remaining consistent with the invention. For example, signal molecule can be attached to the dendrimer, before, during, or after attachment of the probe binding sequence; the RNA probes can be exposed to the target nucleic acid sequence before, during, or after hybridization to the dendrimer; or so forth.

Similarly, the above steps can modified or steps can be deleted, also consistent with the invention. For example, an existing cDNA library can be utilized wherein all of the strands already include a particular existing sequence that serves as the capture sequence; cDNA molecules can be modified to incorporate the capture sequence therein; or so forth.

### BRIEF DESCRIPTION OF THE DRAWINGS

FIG. 1 is a diagram showing preparation of an RNA probe for use with the current invention, using methods known in the art.

FIG. 2 is a diagram showing a blot assay using dendritic capture reagents in accordance with the present invention.

FIG. 3 is a diagram showing a method of microarray detection using dendritic reagents in accordance with the present invention.

### DETAILED DESCRIPTION OF THE INVENTION AND THE PREFERRED EMBODIMENTS

In accordance with the invention, a method is provided for detecting nucleic acids without incorporating labeled nucleotides into the target or the probe nucleic acid strands. Pursuant to the methods, capture sequences are attached to one of the nucleic acid strands (preferably the probe strand) and those capture sequences are hybridized to a signal carrying molecule. In the preferred embodiment, the signal carrying molecule is a dendritic molecule, such as 3DNA™ available from Genisphere Inc. and Datascope Corp. of Montvale, N.J. Such dendritic molecules (“dendrimers”) are described in U.S. Pat. Nos. 5,175,270 and 5,484,904, and in Nilsen et al., Dendritic Nucleic Acid Structures, J. Theor. Biol., 187, 273-284 (1997), all of which are fully incorporated herein by reference.

Further in accordance with the invention, probe nucleic acid is provided which includes a portion that will potentially hybridize to the target molecule, and further includes a segment that will not hybridize. The probes are prepared using methods known in the art.

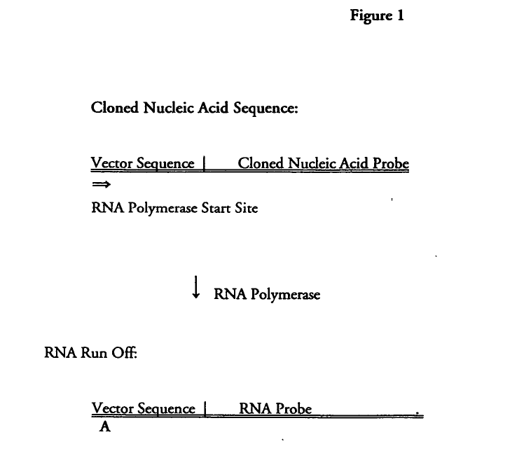

For example, in one embodiment, suitable nucleic acid probes can be generated by taking a vector containing the cloned DNA fragment to be used as the RNA probe, linearizing it via the use of restriction enzymes, and then preparing an RNA run off from that fragment by transcribing it using T₇, T₃, or SP₆ RNA polymerase. The RNA so produced contains a segment of vector nucleic acid sequence that is not part of the cloned probe that binds to target, as shown in FIG. 1. The vector sequence designated “A” in this example will subsequently serve as the dendritic capture sequence. Any desired vector sequence can be utilized, provided the sequence will not be complementary to a target sequence, or so forth. The transcribed RNA portion of the run off will serve as the probe for binding to target molecules.

Subsequently, DNA dendrimers are prepared using known methods. See e.g., U.S. Pat. Nos. 5,175,270 and 5,484,904, and in Nilsen et al., Dendritic Nucleic Acid Structures, J. Theor. Biol., 187, 273-284 (1997), all of which are fully incorporated herein by reference. An oligonucleotide sequence complementary to the capture sequence is attached to an outer arm of the dendritic molecule (this complementary sequence being referred to as the probe binding sequence or A′) . In other words, this probe binding sequence A′ is complementary to the vector sequence A described above and shown in FIG. 1.

The probe binding sequence can be attached via ligation or hybridization of an oligonucleotide to the outer surface arms of the dendritic molecules as previously described in U.S. Pat. Nos. 5,175,270 (“the '270 patent”) and 5,484,904 (“the '904 patent”), both of which are fully incorporated herein by reference. Signal molecules are also attached to the dendrimers (before, during or after attachment of the probe binding sequence) via oligonucleotides that hybridize to the outer surface dendritic arms as previously described in the '270 patent and the '904 patent.

In the various embodiments of the invention, the probe is hybridized to the target sequence and the dendritic molecule is attached to the probe (via hybridization of the probe binding sequence to the capture sequence). These hybridizations can be conducted on in any desired format (solid-phase hybridization, liquid phase, or so forth).

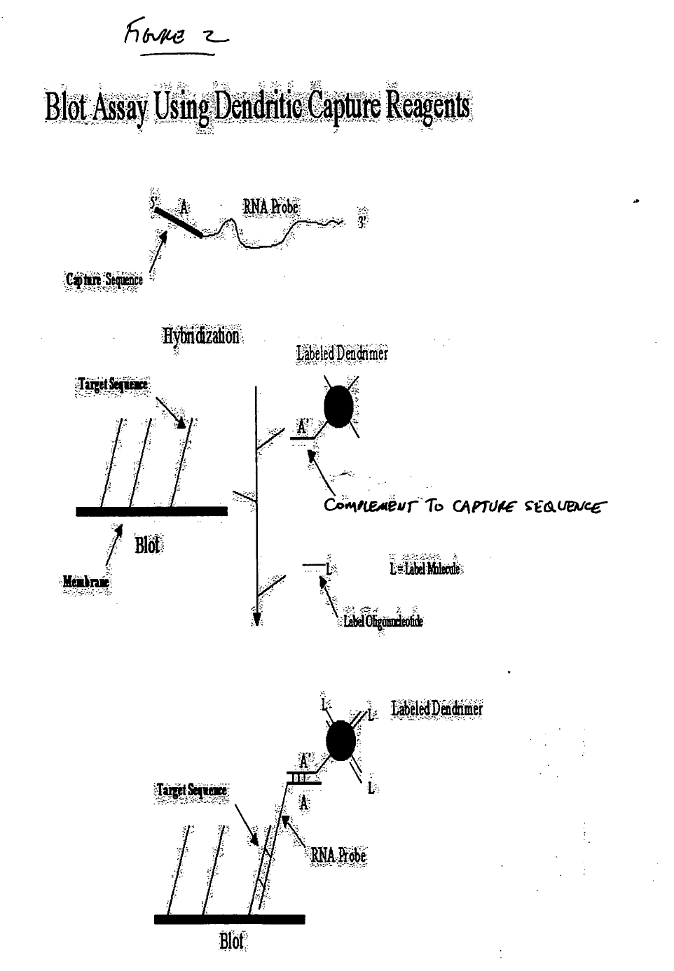

In one embodiment of the invention, for example, as shown in FIG. 2, the hybridizations are conducted using classical blot assays. For this type of assay, cellular nucleic acid DNA or RNA (the target) is separated by size on an agarose gel and is subsequently transferred (blotted) to a solid support (known as a membrane), by methods familiar to those skilled in the art.

In accordance with the invention, the blot (containing the target sequences) is then combined with the RNA probe (e.g. the RNA runoff probe described above) and dendritic DNA. If the dendimers have already been labeled with signal molecule, detection of the signal molecules can be conducted. Otherwise, signal molecules can be added before, during, or after the hybridization of the dendrimer to the probe. These signal molecules can be biotin, oligooxigenin, ³²P or other suitable molecules, e.g. using the methods of the '270 patent and the '904 patent or Nilsen reference, or other methods as currently known in the art. Since the probe binding sequence (A′) on the dendrimer hybridizes to the complementary capture sequence on the probe, the detection of signal from the dendrimer indicates the presence of a hybridized complex of probe and target nucleic acids on the blot.

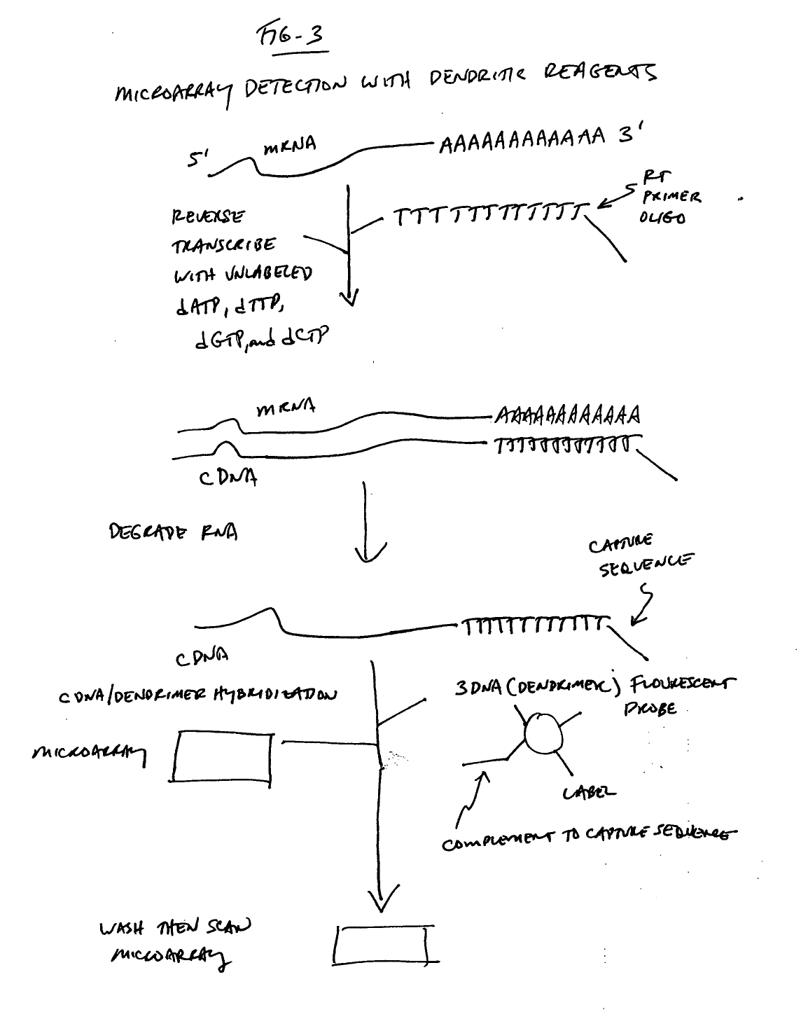

In a further embodiment of the invention, the hybridizations can be conducted using fluorescent based microarrays (e.g. for RNA expression analysis), as shown in FIG. 3.

In this embodiment, RNA molecules are provided for reverse transcription wherein the molecules have a poly A tail. The RT primer used for the transcription operation is a bifunctional oligonucleotide—in other words, it is composed of both a 3′ oligo dT sequence and a 5′ dendrimer binding sequence (the capture sequence).

The 3′ oligo dT sequence serves as a primer for the RNA copying enzyme, reverse transcriptase, and can range in length from 15 to 30 nucleotides. This oligo dT sequence will hybridize to the 3′ poly A tail of RNA and will serve as the starting point for the synthesis of DNA copies (cDNA) of the RNA messages found in the sample. The poly A RNA can be part of the total cellular RNA, or purified by published protocols or available kits, or so forth. Or, alternatively, in the case of RNA without poly A tails, those tails can be added to the RNA probes in the sample. Reverse transcription from a population of the total cellular RNA will yield a copy of the entire (poly A) population.

After reverse transcription of the cDNA strands, the RNA is degraded, leaving cDNA probes having a 5′ capture sequence that came from the RT primer. This 5′ capture sequence, which is incorporated into each cDNA, is provided for the subsequently binding of dendrimer molecule to the cDNA probe.

As discussed previously, the dendrimers are prepared by attaching two oligonucleotides to the outer surface arms of the core dendrimer structure. The first oligonucleotide is the probe binding sequence, a sequence which is complementary to the capture nucleic acid sequence present in the probe. The probe binding sequence can be attached by either ligation or hybridization followed by cross-linking.

This probe binding sequence will hybridize to and capture the 5′ end of a reverse transcription primer, as discussed above. Thus, the hybridization of the capture sequence on the probe to the complementary probe binding sequence on the dendrimer bridges the signal carrying dendrimer to the cDNA. This dendritic probe binding sequence is designed to avoid any cross-hybridization with the dendrimer core reagents and other published nucleic acid sequences, such as those found in public domain databases.

The second oligonucleotide on the dendrimer is the “label” oligonucleotide which is a signal molecule (e.g. a fluorescent dye molecule). This signal molecule is attached to either the 3′ end, the 5′ end, both ends, or one or more internal nucleotide bases of the dendrimer.

The signal molecule (e.g. fluorescent oligonucleotide) is hybridized and cross-linked to a complementary dendrimer binding arm. Any fluorescent dye that can be coupled to DNA can be attached to dendrimers for this application. Examples include Cy3™, Cy5™, fluorescent, Oregon Green™, the Alexa™ series dyes, and the BODIPY™ dyes, among others. Each dendrimer reagent is labelled with at least 100 individual fluorescent molecules of the same type.

Once the cDNA molecules have been prepared from the poly A RNA, they can be applied to a microarray by a typical hybridization reaction. As discussed above, a microarray generally consists of a substantially planar substrate coated with a grid of tiny spots of about 20 to 100 microns in diameter; each spot or feature contains millions of copies of a short sequence of DNA or nucleotides. A computer keeps track of each sequence at a predetermined feature. Any cDNA molecules complementary to strands of nucleic acid at a specific location on the array will hybridize to those strands of nucleic acid and remain immobile.

Excess RT primer and unbound cDNA are then washed away. Next, the signal carrying dendrimer reagent discussed above, which has the complementary probe binding sequence is applied to the probe. When dendrimer molecule is subsequently added to the probe, the cDNA 5′ probe capture sequence will hybridize to a complementary probe binding sequence on the dendrimer. Hybridization of dendrimer to probe which is hybridized to target nucleic acid therefore allows signal detection (via the signal detection molecules of the dendrimer) without the need to label probe or target with radioactive or fluorescent signal molecules or so forth. The array is then washed to remove unbound fluorescent dendrimer and scanned using commercially available hardware and software to develop the signal.

Several examples of experimental protocols for use in conjunction with the invention, are provided as follows:

### EXAMPLE 1

With reference to FIGS. 1 and 2, a method for nucleic add detection using RNA Run-off probes and blot assays is as follows:

Preparation of RNA Run-off probes

In vitro transcriptions reactions were prepared and run as described for the MAXIscript kit (Ambion, Austin, Tex.). Briefly, 250 ng (2.0Fl) of plasmid p-Tri-Cyclin-D2 (Ambion, Austin, Tex.) was combined and mixed with 2FL of 10× Transcription Buffer, 1Fl each of dATP, dCTP, dTTP and dGTP in a final volume of 17Fl in a 1.5 ml microfuge tube. One microliter (1.0Fl) of RNase Inhibitor was added to prevent the RNA product from degrading after synthesis. T7 RNA polymerase (2.0Fl) was added and the tube was mixed and briefly microfuged. The reaction mixture was incubated at 37° C. for 45 minutes to product the RNA Run-off product. The reaction was terminated by heating to 65-70° C. for 15 minutes. The DNA template was removed by digesting adding 1.0 ul of RNase-free DNase I and incubating the mixture at 37° C. for 15 minutes. The DNase digestion was stopped by adding 1 ul of 0.5M EDTA, pH=8.0. The RNA Run-off probe was gel purified using a 10% TBE-Urea gel (Invitrogen, Carlsbad, Calif.) and eluting the probe into 1.5 mls of RNase free 50 mM Tris-HCl, 10 mM EDTA, pH=8.0. The probe was stored at −70° C. until use. This RNA Run-off probe contained DNA sequence corresponding to the Cyclin D2 gene as well as a short sequence (approximately 50 bases) that was derived from the DNA sequence of the plasmid located between the RNA polymerase start site and the cloned Cyclin D2 gene sequence.

Preparation of 3DNA″ Dendrimers

A Cyclin D2 capture dendrimer reagent was prepared by ligating an oligonucleotide that is complementary to the short sequence of nucleic acid between the RNA start site and the cloned Cyclin D2 gene sequence of the RNA Run-off probe to DNA dendrimer reagents by standard methods. This dendrimer attached oligonucleotide sequence when mixed with the RNA Run-off would hybridized with the complementary sequence on the RNA Run-off and link it to the 3DNA dendrimer reagent.

Southern Blot Assay

A Southern blot was prepared using standard methods using dilutions of EcoRI restricted Human Genomic DNA. Briefly, samples of restricted genomic DNA equal to 5Fg, 1 ug, 0.2Fg, and 0.04Fg were separated by size on a 1% agarose gel and is subsequently transferred (blotted) using the standard method to a 6 cm by 20 cm piece of Hybond-N membrane (Amersham Pharmacia Biotech, Piscataway, N.J.). The genomic DNA was fixed to the membrane by UV cross-linking and the membrane (Southern blot) was transferred into a hybridization bag. Ten milliliters (10 mls) of ExpressHyb″ (Clontech, Palo Alto, Calif.) was added to the hybridization bag. The hybridization bag was sealed mixed and transferred into a 65° C. water bath for 30 minutes to prehybridize the membrane.

32P (kinase) labeling of Dendrimer Label Oligonucleotides

In a microfuge 1Fg (5Fl) of each of the oligonucleotides that bind to the free single stranded arms of dendrimer reagents was combined with 10 ul of 10× kinase buffer, 100 FCi of gamma 32P ATP (NEN, Boston, Mass.), 1Fl of T4 polynucleotide kinase (Amersham Pharmacia Biotech, Piscataway, N.J.) in a final volume of 100 ul. The contents were mixed and incubated at 37° C. for 1 hour. The reaction was stopped by adding 2 ul of 0.5M EDTA, pH=8.0. The free unincorporated label nucleotide was removed by G-50 chromatography using Quick Spin Columns (Roche, Indianapolis, Ind.).

During the prehybridization 100Fl of the gel purified Cyclin D2 RNA Run-off probe (1/15th) was combined with 10Fl (200 ng) of Cyclin D2 capture dendrimer in 0.5 mls of ExpressHyb″ and 10Fl of 32P labeled oligonucleotides. At the end of the 30 minutes, membrane prehybridization step, this mixture was added to the hybridization bag containing the Southern blot membrane. The Southern blot was hybridized overnight (−16 hours) at 65° C. On the following morning the hybridization bag containing the Southern blot was cut open ant the membrane was transferred into 500 mls of 2×SSC, 1% SDS prewarmed to 65° C. and washed for 30 minutes. The membrane was transferred into prewarmed 2×SSC, 1% SDS and washed 30 minutes at 65° C. This wash step was repeated. The membranes were transferred into 0.5×SSC, 0.1% SDS and washed at 65° C. for 30 minutes. This wash step was repeated. The membrane was then drained of excess wash buffer and wrapped in plastic wrap, exposed to a Phosphor Screen and read using a STORM instrument (Molecular Dynamics, Sunnyvale, Calif.). A band of radioactive signal was observed at the position on the membrane corresponding to the Cyclin D2 gene.

### EXAMPLE 2

With reference to FIG. 3, a method for detection and assay on a microarray is described below.

Microarray Preparation

A microarray was prepared as directed by the manufacturer or by customary procedure protocol. The nucleic acid sequences comprising the DNA or gene probes were amplified using known techniques in polymerase chain reaction, then spotted onto glass slides, and processed according to conventional procedures.

Preparation and Concentration of Target Nucleic Acid Sequences Sample, or cDNA

The target nucleic acid sequences, or cDNA was prepared from total RNA or poly(A)+RNA extracted from a sample of cells. It is noted that for samples containing about 10 to 20 μg of total RNA or 500-1000 ng of poly(A)⁺ RNA, ethanol precipitation is not required and may be skipped, because the cDNA is sufficiently concentrated to perform the microarray hybridization. In a microfuge tube, 0.25 to 5 μg of total RNA or 12.5 to 500 ng of poly(A)⁺ RNA was added with 3 μL of Cy3® or Cy5® RT primer (0.2 pmole) and RNase free water for a total volume of 10 μL to yield a RNA-RT primer mixture. The resulting mixture was mixed and microfuged briefly to collect contents in the bottom of the microfuge tube. The collected contents was then heated to 80° C. for about ten (10) minutes and immediately transferred to ice. In a separate microfuge tube on ice, 4 μL of 5×RT buffer, 1 μL of dNTP mix, 4 μL RNase free water, and 1 μL of reverse transcriptase enzyme (200 Units) were combined to yield a reaction mixture. The reaction mixture was gently mixed and microfuged briefly to collect contents in the bottom of the microfuge tube. 10 μL of the RNA-RT primer mixture and 10 μL of the reaction mixture, was mixed briefly and incubated at 42° C. for two hours. The reaction was terminated by adding 3.5 μL of 0.5 M NaOH/50 mM EDTA to the mixture. The mixture was incubated at 65° C. for ten (10) minutes to denature the DNA/RNA hybrids and the reaction was neutralized with 5 μL of 1 M Tris-HCl, pH 7.5. 38.5 μL of 10 mM Tris, pH 8.0, 1 mM EDTA was then added to the neutralized reaction mixture. (The above steps may be repeated replacing the 3 μL of Cy3® RT primer (0.2 pmole) with 3 μL of Cy5® RT primer (0.2 pmole) for preparing dual channel expression assays whereby the prepared Cy3® and Cy5® cDNA mixture are mixed together with 10 μL of 10 Tris, pH 8.0, 1 mM EDTA, to yield a reaction mixture for processing in the following steps.)

2 μL of a carrier nucleic acid (10 mg/mL linear acrylamide) was added to the neutralized reaction mixture for ethanol precipitation. 175 μL of 3M ammonium acetate was added to the mixture and then mixed. Then, 625 μL of 100% ethanol was added to the resulting mixture. The resulting mixture was incubated at −20° C. for thirty (30) minutes. The sample was centrifuged at an acceleration rate greater than 10,000 g for fifteen (5) minutes. The supernatant was aspirated and then 330 μL of 70% ethanol was added to the supernatant, or cDNA pellet. The cDNA pellet was then centrifuged at an acceleration rate greater than 10,000 g for 5 minutes, was then remove. The cDNA pellet was dried (i.e., 20-30 minutes at 65° Celsius).

Hybridization of cDNA/Dendrimer Probe Mixture to Microarray

The DNA hybridization buffer was thawed and resuspended by heating to 65° C. for ten (10) minutes. The hybridization buffer comprised of 40% formamide, 4×SSC, and 1% SDS. The buffer was mixed by inversion to ensure that the components were resuspended evenly. The heating and mixing was repeated until all of the material was resuspended. A quantity of competitor DNA was added as required (e.g. 1 μg COT-1-DNA, and 0.5 μg polydT). The cDNA was resuspended in 5.0 μL of sterile water.

In a first embodiment, single channel analysis, 2.5 μL of one type of 3DNA® reagent (Genisphere, Inc., Montvale, N.J.) (Cy3 or Cy5) was added to the resuspended cDNA along with 12.5 μL of a DNA hybridization buffer (containing 40% formamide). In an alternative embodiment, for dual channel analysis, 2.5 μL of two types of 3DNA® reagents, Cy3 and Cy5 specifically labeled dendrimers, were added to the resuspended cDNA along with 10 μL of a DNA hybridization buffer. In a further embodiment of multiple channel analysis (with three or more channels), 2.5 μL of three or more types of 3DNA® reagents, Cy3, Cy5, and one or more prepared using another label moiety, were added to the resuspended cDNA along with 10 μL of a DNA hybridization buffer.

For larger hybridization buffer volumes, additional DNA hybridization buffer may be added to the required final volume. It is noted that hybridization buffer volumes greater than 35 μL may also require additional 3DNA® reagents.

The DNA hybridization buffer mixture was incubated at about 45-50° C. for about 15 to 20 minutes to allow for prehybridization of the capture sequence on the cDNA to the complementary sequence on the 3DNA® reagents. The prehybridized mixture was then added to the microarray and then incubated overnight at 55° C. At this stage the cDNA was hybridized to the gene probes.

Post Hybridization Wash

The microarray was briefly washed to remove any excess dendrimer probes. First, the microarray was washed for 10 minutes at 55° C. with 2×SSC buffer, 0.2% SDS. Then the microarray was washed for 10 minutes at room temperature with 2×SSC buffer. Finally the microarray was washed for 10 minutes at room temperature with 0.2×SSC buffer.

Signal Detection

The microarray was then scanned as directed by the scanner's manufacturer for detecting, analyzing, and assaying the hybridization pattern.

### EXAMPLE 3

A alternative method for detection and assay on a microarray is described below. This method includes the use of a spin column assembly for reducing protocol time and number of steps, and for increasing signal strength.

Microarray Preparation

A microarray was prepared as directed by the manufacturer or by customary protocol procedures. The nucleic acid sequences comprising the DNA or gene probes were amplified using known techniques in polymerase chain reaction, then spotted onto glass slides, and processed according to conventional procedures.

Preparation and Concentration of Target Nucleic Acid Sequences, or cDNA

The target nucleic acid sequences, or cDNA was prepared from total RNA or poly(A)+RNA extracted from a sample of cells. In a microfuge tube, 0.25 to 5 μg of total RNA or 12.5 to 500 ng of poly(A)+RNA was added with 1 μL of Cy3® or Cy5® RT primer (5 pmole) and RNase free water for a total volume of 10 μL to yield a RNA-RT primer mixture. The resulting mixture was mixed and microfuged briefly to collect contents in the bottom of the microfuge tube. The collected contents was then heated to 80° C. for about ten (10) minutes and immediately transferred to ice. In a separate microfuge tube on ice, 4 μL of 5×RT buffer, 1 μL of dNTP mix, 4 μL RNase free water, and 1 μL reverse transcriptase enzyme (200 Units) were combined to yield a reaction mixture. The reaction mixture was gently mixed and microfuged briefly to collect contents in the bottom of the microfuge tube. 10 μL of the RNA-RT primer mixture and 10 μL of the reaction mixture was mixed together and incubated at 42° C. for two hours. The reaction was terminated by adding 3.5 μL of 0.5 M NaOH/50 mM EDTA. The mixture was incubated at 65° C. for ten (10) minutes to denature the DNA/RNA hybrids. The reaction was neutralized by the addition of 5 μL of 1 M Tris-HCl, pH 7.5 to the mixture. 71 μL of 10 mM Tris, pH 8.0, 1 mM EDTA was added to the neutralized reaction mixture. (The above steps may be repeated replacing the 1 μL of Cy3® RT primer (5 pmole) with 1 μL of Cy5® RT primer (5 pmole) for preparing dual channel expression assays whereby the prepared Cy3® and Cy5® cDNA mixture are mixed together with 42 μL of 10 mM Tris, pH 8.0, 1 mM EDTA, to yield a reaction mixture for processing in the following steps.)

cDNA Purification: Removal of Excess RT Primer via a SC Spin Column Assembly

The spin column was inverted several times to resuspend the media and to create an even slurry in the column. The top and bottom caps were removed from the spin column. A microfuge tube was obtained and the bottom tip of the microfuge tube, was snipped off or punctured. One end of the spin column was placed into the punctured microfuge tube, then the punctured microfuge tube was placed into a second, intact microfuge tube, or collection tube. The assembled spin column was then placed into a 15 μL centrifuge tube with the microfuge tube end first. The spin column was centrifuged at about 1000 g for about 3.5 minutes after reaching full acceleration. The spin column was checked to ensure that the column was filly drained after centrifugation and that the end of the spin column was above the liquid line in the collection tube. The collection tube contained about 2 to 2.5 mL of clear buffer voided from the spin column. The resin appeared nearly dry in the column barrel, and well packed without distortions or cracks. If the end of the spin column had been immersed in the liquid portion, the spin column would have been discarded and the above steps repeated with a fresh spin column. The spin column was at that point, prepared to remove the excess RT primer in the neutralized reaction mixture.

The drained spin column was removed and a new 1.0 mL collection tube was placed on top of the buffer collection tubes already in the 15 mL centrifuge tube. The voided buffer was discarded. The drained spin column was placed into the new collection tube. 100 μL of the neutralized reaction mixture containing the cDNA was loaded directly into the center of the spin column media The spin column assembly was centrifuged at 10,000×g for about 2.5 minutes upon reaching full acceleration. The eluate collected in the new collection tube was then recovered. About 10 percent of the original reaction mixture was recovered. The eluate comprised the cDNA probe.

2 μL of a carrier nucleic acid (10 mg/mL linear acrylamide) was added to the eluate for ethanol precipitation. 250 μL of 3M ammonium acetate was added to the mixture and mix. Then, 875 μL of 100% ethanol was added to the mixture. The resulting mixture was incubated at −20° C. for thirty (30) minutes. The sample was centrifuged at an acceleration rate greater than 10,000×g for fifteen (15) minutes. The supernatant was aspirated and 300 μL of 70% ethanol was added to the supernatant, or the cDNA pellet. The cDNA pellet was then centrifuged at an acceleration rate greater than 10,000×g for 5 minutes. The supernatant was then removed. The cDNA pellet was dried (i.e. 20-30 minutes at 65° Celsius).

Hybridization of cDNA/Dendrimer Probe Mixture to Microarray

The DNA hybridization buffer was thawed and resuspended by heating to 65° C. and maintained at 65° C. for ten (10) minutes. The hybridization buffer comprised of 40% formamide, 4×SSC, and 1% SDS. The buffer was mixed by inversion to ensure that the components were resuspended evenly. The heating and mixing was repeated until all the material was resuspended. A quantity of competitor DNA (e.g. 1.0 μg COT-1-DNA, and 0.5 μgpolydT) may be added, if required. The cDNA was resuspended in 5.0 μL of sterile water.

In a first embodiment, single channel analysis, 2.5 μL of one type of 3DNA® reagent (Genisphere, Inc., Montvale, N.J.) (Cy3 or Cy5) was added to the resuspended cDNA along with 12.5 μL of a DNA hybridization buffer (containing 40% formamide). In an alternative embodiment, for dual channel analysis, 2.5 μL of two types of 3DNA® reagents, Cy3 and Cy5 specifically labeled dendrimers, were added to the resuspended cDNA along with 10 μL of a DNA hybridization buffer. In a further embodiment of multiple channel analysis (with three or more channels), 2.5 μL of three or more types of 3DNA® reagents, Cy3, Cy5, and one or more prepared using another label moiety, were added to the resuspended cDNA along with 10 μL of a DNA hybridization buffer.

For larger hybridization buffer volumes, additional amounts of the DNA hybridization buffer may be added to reach the required final volume. It is also noted that hybridization buffer volumes greater than 35 μL may also require additional 3DNA® reagents. The DNA hybridization buffer mixture was incubated at a temperature of about 45-50° C. for about 15 to 20 minutes to allow for the prehybridization of the cDNA to the 3DNA® reagents or dendrimer probes. At this stage, the dendrimer probes of the 3DNA® reagent hybridized with the capture sequence on the cDNA After 20 minutes, the DNA hybridization buffer was then added to the microarray. The microarray and the DNA hybridization buffer were covered and incubated overnight in a humidified chamber at a temperature of about 55° C. At this stage, the cDNA was hybridized to the gene probes.

Post Hybridization Wash

The microarray was briefly washed to remove any excess dendrimer probes. First, the microarray was washed for 10 minutes at 55° C. with 2×SSC buffer, containing 0.2% SDS. Then, the microarray was washed for 10 minutes at room temperature with 2×SSC buffer. Finally, the microarray was washed for 10 minutes at room temperature with 0.2×SSC buffer.

Signal Detection

The microarray was then scanned as directed by the scanner's manufacturer for detecting, analyzing, and assaying the hybridization pattern.

Although several illustrations of the invention are provided above, the present invention can also be used in conjunction with any of the inventions described in PCT Application No. PCT/US01/07477 filed 8 Mar. 2001, PCT Application No. PCT/US01/22818 filed 19 Jul. 2001, and/or U.S. Provisional Application Serial No. 60/316,116 filed Aug. 31, 2001, all of which are filly incorporated herein by reference.

Having described this invention with regard to specific embodiments, it is to be understood that the description is not meant as a limitation since further embodiments, modifications and variations may be apparent or may suggest themselves to those skilled in the art. It is intended that the present application cover all such embodiments, modifications and variations.

What is claimed is:

1. A method for detection of nucleic acids, comprising the steps of:
a) linearizing a vector containing vector nucleic acid and a cloned nucleic acid;
b) transcribing said fragment to produce a transcribed nucleic acid comprising a segment derived from said vector nucleic acid and a segment derived from said cloned nucleic acid, said segment derived from said vector nucleic acid comprising a capture sequence, and said segment derived from said cloned nucleic acid comprising a probe;
c) providing dendrimer molecules, said dendrimer molecules comprising a nucleic acid sequence complementary to said capture sequence, and further comprising a signal molecule;
d) providing a membrane comprising target nucleic acid; and,
e) conducting a blot assay using said transcribed nucleic acids, said dendrimer molecules, and said membrane.

2. A method as claimed in claim 1, further comprising the step of fixing genomic DNA to said membrane.

3. A method as claimed in claim 1, further comprising the step of preparing said dendrimer molecule by attaching to a dendrimer reagent said nucleic acid sequence complementary to said capture sequence.

4. A method for detection of nucleic acids, comprising the steps of:
a) linearizing a vector containing vector nucleic acid and a cloned nucleic acid;
b) transcribing said fragment to produce a transcribed nucleic acid comprising a segment derived from said vector nucleic acid and a segment derived from said cloned nucleic acid, said segment derived from said vector nucleic acid comprising a capture sequence, and said segment derived from said cloned nucleic acid comprising a probe;
c) providing dendrimer molecules, said dendrimer molecules comprising a nucleic acid sequence complementary to said capture sequence, and further comprising a signal molecule;
d) hybridizing said probe to a target nucleic acid; and
e) hybridizing said dendrimer to said capture sequence.

5. A method as claimed in claim 4, further comprising the step of preparing said dendrimer molecule by attaching to a dendrimer reagent said nucleic acid sequence complementary to said capture sequence.
